(19) United States
(12) Patent Application Publication
(10) Pub. No.: US 2024/0016765 A1
(43) Pub. Date: Jan. 18, 2024

(54) Use of an alkaloid compound in the preparation of products for the prevention and/or treatment of cardiac damage

(71) Applicant: NORTHWEST INSTITUTE OF PLATEAU BIOLOGY, CHINESE ACADEMY OF SCIENCES, Xining City (CN)

(72) Inventors: Honglun Wang, Xining City (CN); Wenna Zhou, Xining City (CN); Na Hu, Xining City (CN)

(21) Appl. No.: 18/476,247

(22) Filed: Sep. 27, 2023

Related U.S. Application Data
(63) Continuation of application No. PCT/CN2022/078277, filed on Feb. 28, 2022.

(30) Foreign Application Priority Data
Mar. 29, 2021 (CN) 202110334616.2

(51) Int. Cl. A61K 31/165; A61K 31/704; A61P 9/00; A61K 9/20; A61K 47/26; A61K 9/00

(52) U.S. Cl. CPC A61K 31/165; A61K 31/704; A61P 9/00; A61K 9/2018; A61K 9/2059; A61K 9/2054; A61K 9/2013; A61K 9/2009; A61K 47/26; A61K 9/0019

(57) ABSTRACT

The present invention discloses the use of an alkaloid compound in the preparation of products for the prevention and/or treatment of cardiac damage. Activity studies are conducted on the alkaloid compound, and it is verified that the alkaloid compound provided by the present invention is capable of inhibiting apoptosis induced by cardiotoxic effects. Specifically, it can inhibit cardiomyocyte apoptosis induced by the anthracycline DOX by down-regulating the expression level of p-JNK and/or Cleaved-Caspase-3, thus preventing and/or treating cardiomyocyte toxicity. The alkaloid compound can be used in the preparation of drugs for the prevention and/or treatment of diseases related to cardiac damage.

### TECHNICAL FIELD

The present invention relates to the field of pharmaceutical uses of alkaloid compounds, and specifically to the use of an alkaloid compound in the preparation of products for the prevention and/or treatment of cardiac damage.

### BACKGROUND TECHNOLOGY

Seabuckthorn (Latin name: Hippophae rhamnoides Linn.), a deciduous shrub of the family Elaeaceae and genus Sea Buckthorn, is widely used for soil and water conservation as it is drought tolerant, resistant to wind and sand, and can survive on saline-alkaline soil. Seabuckthorn is planted in large quantities in northwestern China for desert greening.

Seabuckthorn is rich in nutrients and contains a variety of vitamins, flavonoids, triterpenoids, oils and fatty acids, phenols, volatile oils, trace elements, phospholipids, 5-hydroxytryptamine and other active substances as well as various amino acids and proteins needed by the human body.

Seabuckthorn seeds are often used to extract oil to make seabuckthorn oil, which contains 206 kinds of active substances beneficial to human body, of which 46 kinds having biological activities. It contains a large amount of vitamin E, vitamin A, flavonoids and so on. After oil extraction, a large amount of seabuckthorn seed dreg is produced, which is often discarded as waste or produced into dry powder as feed, which is very cheap. If more utilization value of seabuckthorn seed dreg can be developed, it will bring great economic significance.

In addition, many of the active substances contained in seabuckthorn have not yet been isolated or identified. If chemical components and their pharmacological effects in seabuckthorn can be studied more deeply and minutely and the active mechanism can be explored, it is expected to develop more natural plant-derived medicines that can treat diseases, with good safe and better efficacy.

### CONTENT OF THE INVENTION

Currently, many drugs used in the treatment process may have cardiotoxic side effects, especially cytotoxic agents used in chemotherapy for malignant tumors, which can cause serious heart toxicity, adversely affecting patients' prognosis and recovery.

Anthracyclines are commonly reported to be cardiotoxic, but such drugs are widely used in the clinical practices due to their better antitumor activity. Anthracyclines include doxorubicin, daunorubicin, aclacinomycin, pharmorubicin, pirarubicin, idarubicin, and mitoxantrone etc. Among them, doxorubicin (once known as adriamycin) is one of the commonly used broad-spectrum antitumor drugs in clinical practices, which exhibits good efficacy in the treatment of malignant tumors, and has been widely used in the treatment of solid tumors, leukemias, lymphomas, breast cancers, and other tumors since the early 1960s. Doxorubicin has high affinity to myocardial tissues, and its main mechanism of anti-tumor action is that its anthracene plane can be directly embedded between DNA base pairs, interfering with the transcription process and preventing the synthesis of mRNA to inhibit both the synthesis of DNA and the synthesis of RNA, therefore, it has an effect on the various stages of the cell cycle, and belongs to the non-specific drugs of the cell cycle; however, its dose-dependent cardiotoxicity has been a major concern in oncology treatment practices since the development of drugs for clinical use. This side effect is manifested by a high incidence (up to 11% to 30%) of congestive heart failure when the therapeutic dose of the drug exceeds 550 mg/m², with a mortality rate as high as 50%-60%. The mechanism of adriamycin-induced cardiomyopathy is different from its antitumor mechanism of action, but still not fully understood. However, many studies have shown that adriamycin induces oxidative stress, calcium overload, mitochondrial damage, and inhibition of cardiomyocyte-specific gene expression in cardiomyocytes, etc, thereby inducing apoptosis, necrosis, myocardial fibrosis, and making the myocardial contractility reduced, which ultimately leads to ventricular remodeling and congestive heart failure.

Therefore, it is of great importance to develop substances that can reduce the damage to the heart caused by cardiotoxic substances and resist cardiotoxicity.

In response to the above problem, the present invention provides the use of alkaloid natural compound I (4-[(E)-p-coumaroylamino]butan-1-ol) extracted from seabuckthorn seed dreg in the preparation of products for the treatment and/or prevention of cardiac damage. The compound I described here has the potential for preventing and/or treating cardiac damage. Its mechanism of action involves downregulating the expression levels of p-JNK and/or Cleaved-Caspase-3, thereby inhibiting cardiomyocyte apoptosis induced by anthracycline drug DOX and alleviating cardiotoxic effects. Therefore, this compound can be used for the development of products for the prevention and/or treatment of cardiac injury-related diseases.

The present invention provides the use of an alkaloid compound I in the preparation of products for the prevention and/or treatment of cardiac damage, the stated compound I having the following structural formula:

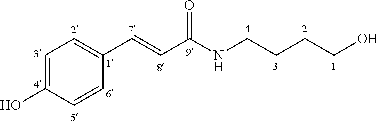 [SMILES: O=C(/C=C/c1ccc(O)cc1)NCCCCO]

Further, the stated cardiac damage is selected from myocardial toxicity and/or apoptosis of cardiomyocytes.

The stated cardiac damage is caused by anthracyclines;

Further, the stated anthracyclines are selected from one or more of doxorubicin, daunorubicin, aclacinomycin, pharmorubicin, pirarubicin, idarubicin, mitoxantrone, preferably doxorubicin.

Further, the stated products are those which delays and/or inhibits the apoptosis of cardiomyocytes.

Further, the stated products are those which can down-regulate the expression levels of Cleaved-Caspase-3 and/or p-JNK.

Further, the stated products are Cleaved-Caspase-3 inhibitor and/or p-JNK inhibitor.

The stated Cleaved-Caspase-3 inhibitor is a product that can cause a decrease in the level of Cleaved-Caspase-3 in vivo; the stated p-JNK inhibitor is a product that can cause a decrease in the level of p-JNK in vivo.

Further, the dosage form of the stated product is selected from tablets, granules, capsules, suppositories, pills, solutions, and suspensions, preferably tablets and solutions; further, the stated solution is selected from injections.

Further, in the specific embodiments of the present invention, of the raw materials of the stated tablets, each 250 mg tablet comprises the following components by weight: 5-20 parts of compound I, 100-200 parts of lactose, 10-25 parts of starch, 50-80 parts of microcrystalline cellulose, 1-10 parts of magnesium stearate, and 1-10 parts of talcum powder; and further, 10 parts of compound I, 150 parts of lactose, 15 parts of starch, 65 parts of microcrystalline cellulose, 5 parts of magnesium stearate, and 5 parts of talc.

Further, in the specific embodiments of the present invention, of the raw materials of the stated solution, each milliliter of the solution comprises the following components by weight: 0.2-1 part of compound I, 30-70 parts of dextrose, 6-12 parts of sodium chloride, and 900-1000 parts of water; and further as 0.5 parts of compound I, 50 parts of dextrose, 9 parts of sodium chloride, and 940.5 parts of water.

The present invention also provides the use of an alkaloid compound I and an anthracycline in the preparation of a combination drug for anti-tumor.

The alkaloid compound I described in the present invention can be made into a combination preparation with pharmaceutically acceptable excipients for use concurrently, separately or in stages during treatment with drugs having cardiotoxic side effects. The combination described herein can be prepared by mixing the alkaloid compound I with an anthracycline to form a single combination preparation, or by preparing two separate preparations of the alkaloid compound I and the anthracycline and then combining them. When the two preparations are made separately, they can be administered concurrently or independently. The frequency and order of application of the two preparations in the stated independent application are not limited, including but not limited to applying the alkaloid compound I first, and then applying the anthracycline drug preparation after a certain interval of time; or applying the anthracycline drug preparation first, and then applying the alkaloid compound I preparation after a certain interval of time; or applying the alkaloid compound I preparation first, followed by an anthracycline preparation at intervals, and then an alkaloid compound I preparation at intervals.

The present invention has the following beneficial effects:

In the present invention, activity studies are conducted on the alkaloid compound I extracted from seabuckthorn seed dreg, and it is verified that the alkaloid compound provided by the present invention is capable of inhibiting myocardial toxicity induced by doxorubicin (DOX). Specifically, it could inhibit cardiomyocyte apoptosis induced by DOX by down-regulating the expression level of p-JNK and/or Cleaved-Caspase-3, thereby, it can be used to prepare products for preventing and/or treating cardiac damage caused by cardiotoxic substances.

### ILLUSTRATION OF THE ACCOMPANYING FIGURES

FIG. 1 showed the effects of alkaloid compound I on the toxicity of H9c2 cardiomyocytes;

FIG. 2 showed the effect of alkaloid compound I on DOX-induced toxicity in H9c2 cardiomyocytes;

FIG. 3 showed the effect of alkaloid compound I on DOX-induced caspase-3 activation in H9c2 cardiomyocytes;

FIG. 4 showed the effect of alkaloid compound I on DOX-induced p-JNK/JNK in H9c2 cardiomyocytes.

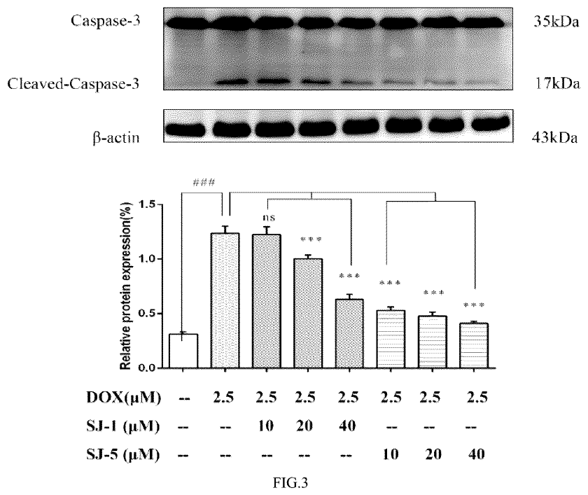

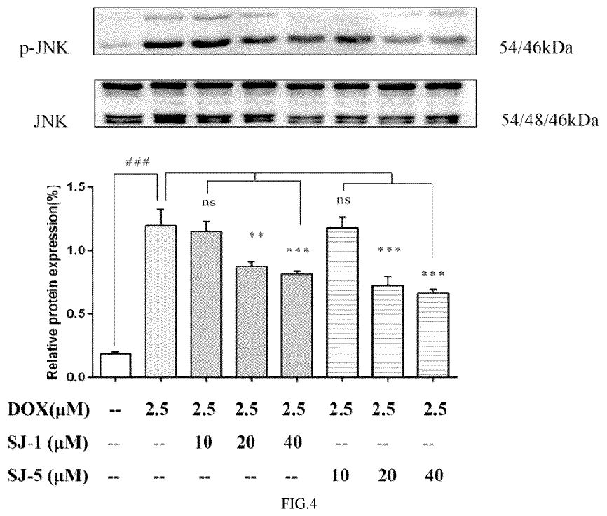

Wherein, SJ-1 is alkaloid compound I in FIGS. 3 and 4; compared with Con group, # indicates P<0.05, ## indicates P<0.01, and ### indicates P<0.001; compared with DOX model group, * indicates P<0.05, ** indicates P<0.01, and *** indicates P<0.001; Mean±SD, n=3.

### SPECIFIC EMBODIMENTS

The technical embodiments of the present invention are described clearly and completely below, and it is obvious that the described embodiments are a part of the embodiments of the present invention and not all of the embodiments. Based on the embodiments in the present invention, all other embodiments obtained by those skilled in the technology without making creative work fall within the scope of protection of the present invention.

The experimental methods in the embodiments of the present invention, if not otherwise specified, are conventional methods; the test materials used, if not otherwise specified, are purchased from conventional biochemical reagent companies, and the data involved in the embodiments are average values.

The structural formula of the alkaloid compound I described in the present invention is as follows:

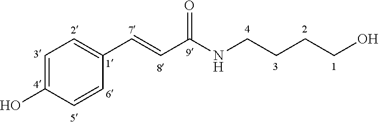 [SMILES: O=C(/C=C/c1ccc(O)cc1)NCCCCO]

Embodiment 1 Test of inhibitory effect of compound I on cardiomyocyte apoptosis in H9c2 cardiotoxicity model

1. Effects of alkaloid compound I on the toxicity of H9c2 cardiomyocytes

H9c2 cells are incubated with different concentrations (0.1 μM, 1 μM, 5 μM, 10 μM, 20 μM, 40 μM, 80 μM, 160 μM) of alkaloid compounds I for 24 h, and then the toxic effects of the alkaloid compounds on cardiomyocytes are evaluated by MTT colorimetric method.

2. Effects of alkaloid compounds I on DOX-induced H9c2 cardiomyocyte toxicity

(1) H9c2 cells are pre-incubated with alkaloid compound I for lh, then add 2.5 μM doxorubicin (DOX), incubate for 24 h. MTT colorimetric assay is used to determine the apoptotic activity of alkaloid compound I on DOX-induced cardiomyocytes and determine the optimal range of the dose concentration.

(2) Incubate H9c2 cells with alkaloid compound I for 1 hour, then add 2.5pIVI DOX and incubate for 24 hours; extract the proteins, proceed with hydrolysis and collect the resulting solution, centrifuge at 12000 rpm for 15 minutes, collect the supernatant and discard the precipitate, store under −80° C. until analysis. Perform SDS-PAGE electrophoresis (Stepl voltage set at 80V for 30 minutes, followed by Step2 voltage set at 120V for 180 minutes), using 5% concentrated gel. Carry out membrane transfer using a PVDF membrane, with a current of 80 mA for 30 minutes. Then, conduct the closure and hybridization by using 5% skim milk powder for 1 hour of closure, followed by overnight incubation with primary antibody, and 1-2 hours of incubation with secondary antibody at room temperature with gentle shaking. Finally, visualize the proteins by using ECL reagent and observe the expression of Caspase-3 and p-JNK/JNK proteins using a gel imaging system.

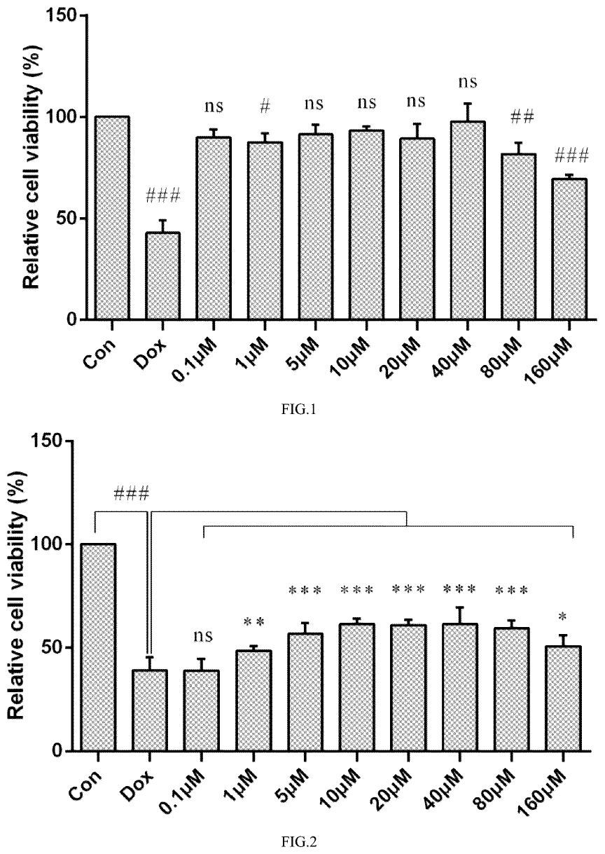

The results in FIG. 1 show that indole alkaloid compound I is not statistically different from the Con group in a certain concentration range (0.1-40 μM), but when the concentration reaches 80 μM, there's significantly different compared with the Con group, suggesting that exceeding this concentration may have some toxic effects on H9c2 cell viability.

The results in FIG. 2 show that compared with the DOX model group, the alkaloid compound groups in different concentration ranges all exhibit inhibition of DOX-induced cardiomyocyte toxicity, and a certain degree of concentration-dependence is observed; compared with the model group, the alkaloid compound group of the concentration range of 5-80 μM had a significant difference or a highly significant difference.

The results in FIG. 3 show that the Cleaved-Caspase-3 expression level is significantly higher in the DOX model group than that in the Con group; the low-, moderate-, and high- dose alkaloid compound I groups (10 μM, 20 μM, and 40 μM) are able to inhibit the elevation of Cleaved-Caspase-3 level induced by DOX to varying degrees compared with the DOX model group, and there is a significant concentration-dependent relationship.

As shown in FIG. 4, the p-JNK expression level is significantly higher in the DOX model group than that in the Con group; There is no statistically significant difference in the p-JNK expression levels between the low-dose alkaloid compound I group (10 μM) and the DOX model group, but a highly significant difference is observed in the moderate-dose (20 μM) and the high-dose (40 μtM) alkaloid compound I groups, and there is an obvious concentration-dependent relationship. This indicates that the alkaloid compounds can down-regulate the expression level of p-JNK in the DOX-induced cardiomyocyte apoptosis model. Combined with the analysis of its effect on the expression level of Cleaved-Caspase-3, it can be concluded that the alkaloid compound provided by the present invention can down-regulate the expression level of p-JNK/Cleaved-Caspase-3 to inhibit DOX-induced cardiomyocyte apoptosis and alleviate the cardiomyocyte toxicity.

In summary, according to the tests, the alkaloid compound I of the present invention can significantly inhibit apoptosis induced by cardiomyotoxic effects and down-regulate the p-JNK/Cleaved-Caspase-3 expression level to achieve preventive and/or therapeutic effects.

The alkaloidal compound I of the present invention is suitable as a drug for mammals, especially humans. It can be used for the prevention and/or treatment of heart damage caused by cardiotoxic doses of drugs or other chemical substances, especially for ameliorating cardiac degeneration and changes, such as inhibiting anthracycline-induced cardiotoxicity and cardiac fibrosis.

The alkaloid compound I of the present invention can be used as a drug, especially as an auxiliary treatment during the administration of drugs with cardiotoxic side effects. Depending on the type of treatment, the substance used and the form of administration, it can be administered intravenously, orally, and the dose may be differennt.

### Embodiment 2

Tablets containing Compound I

The following compositions are used to produce each tablet:

Compound I 10 mg

Lactose 150 mg

Starch 15 mg

Microcrystalline cellulose 65 mg

Magnesium stearate 5 mg

Talc 5 mg

The above materials are mixed, granulated, blended and compressed into a tablet of 250 mg.

### Embodiment 3

Injection containing Compound I

The following compositions are used to produce 1 ml of injectable solution:

Compound I 0.5 mg

Glucose 50 mg

Sodium chloride 9 mg

Purified water 940.5 mg

Dissolve the above solids in purified water and fill the solution aseptically into a 1 ml ampoule.

The aforementioned are only the embodiment of the present invention and do not limit the protection scope of the invention. Any transformation of equivalent processes or equivalent structures made using the description of the present invention, and any direct or indirect application in other related technical fields shall be included in the protection scope of the present invention.

1. A method of the prevention and/or treatment for cardiac damage by administering alkaloid compound Ito patients, the structural formula of which is as follows:

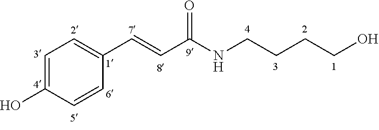 [SMILES: O=C(/C=C/c1ccc(O)cc1)NCCCCO]

2. The method according to claim 1, is characterized in that the stated cardiac damage is selected from cardiotoxicity and/or apoptosis of cardiomyocytes.

3. The method according to claim 1, is characterized in that the stated cardiac damage is due to anthracyclines;
Further, the stated anthracyclines are selected from one or more of doxorubicin, daunorubicin, aclacinomycin, pharmorubicin, pirarubicin, idarubicin, mitoxantrone, preferably doxorubicin.

4. The method according to claim 1, is characterized in that the stated products are those that delay and/or inhibits the apoptosis of cardiomyocytes.

5. The method according to claim 1, is characterized in that the stated products are those which can down-regulate the expression levels of Cleaved-Caspase-3 and/or p-JNK.

6. The method according to claim 5, is characterized in that the stated products are Cleaved-Caspase-3 inhibitor and/or a p-JNK inhibitor.

7. The method according to claim 1, is characterized in that the dosage form of the stated products is selected from tablets, granules, capsules, suppositories, pills, solutions, and suspensions, preferably tablets and solutions; further, the stated solution is selected from injections.

8. The method according to claim 7, is characterized in that, among the raw materials of the stated tablets, each 250 mg tablet comprises the following components by weight: 5-20 parts of compound I, 100-200 parts of lactose, 10-25 parts of starch, - 80 parts of microcrystalline cellulose, 1-10 parts of magnesium stearate, and 1-10 parts of talcum powder; and further, 10 parts of compound I, 150 parts of lactose, parts of starch, 65 parts of microcrystalline cellulose, 5 parts of magnesium stearate, and 5 parts of talc.

9. The method according to claim 7, is characterized in that, among the raw materials of the stated solution, each milliliter of the solution comprises the following components by weight: 0.2-1 part of compound I, 30-70 parts of dextrose, 6-12 parts of sodium chloride, and 900-1000 parts of water; and further as 0.5 parts of compound I, 50 parts of dextrose, 9 parts of sodium chloride, and 940.5 parts of water.

10. An anti-tumor method involves the simultaneous administeration of alkaloid compound I and anthracyclines to patients.
